(19) United States
(12) Patent Application Publication
(10) Pub. No.: US 2004/0127417 A1
(43) Pub. Date: Jul. 1, 2004

(54) N-terminally monopegylated human growth hormone conjugates and process for their preparation

(76) Inventor: Rory F. Finn, Manchester, MO (US)

Correspondence Address: Pharmacia Corporation, Global Patent Department, P.O. Box 1027, Mail Zone MC5, St. Louis, MO, 63141, US

(21) Appl. No.: 10/718,340

(22) Filed: Nov. 20, 2003

Related U.S. Application Data
(60) Provisional application No. 60/427,823, filed on Nov. 20, 2002.

(51) Int. Cl. A61K 38/27; C07K 14/61

(52) U.S. Cl. 514/12; 530/399

(57) ABSTRACT

The present invention provides a chemically modified human Growth Hormone (hGH) prepared by attaching a polyethylene glycol butyraldehyde moiety to the N-terminal phenylalanine of the protein. The chemically-modified protein according to the present invention may have a much longer lasting hGH activity than that of the un-modified hGH, enabling reduced dose and scheduling opportunities.

The present application claims priority under Title 35, United States Code, §119 to U.S. Provisional application Serial No. 60/427,823, filed Nov. 20, 2002, which is incorporated by reference in its entirety as if written herein.

### FIELD OF THE INVENTION

The present invention relates to a chemical modification, including PEGylation, of human Growth Hormone (hGH) and agonist variants thereof by which the chemical and/or physiological properties of hGH can be changed. The PEGylated hGH may have an increased plasma residency duration, decreased clearance rate, improved stability, decreased antigenicity, decreased PEGylation heterogeneity or a combination thereof. The present invention also relates to processes for the modification of hGH. In addition, the present invention relates to pharmaceutical compositions comprising the modified hGH. A further embodiment is the use of the modified hGH for the treatment of growth and development disorders.

### BACKGROUND OF THE INVENTION

Human growth hormone (hGH) is a protein comprising a single chain of 191 amino acids cross-linked by two disulphide bridges and the monomeric form has a molecular weight of 22 kDa. Human GH is secreted by the pituitary gland and which also can be produced by recombinant genetic engineering. hGH will cause growth in all bodily tissues that are capable of growth. Recombinant hGH has been commercially available for several years. Two types of therapeutically useful recombinant hGH preparations are present on the market: the authentic one, e.g. Genotropin™, or Nutropin™ and an analogue with an additional methionine residue at the N-terminal end, e.g. Somatonorm™. hGH is used to stimulate linear growth in patients with hypo pituitary dwarfism also referred to as Growth Hormone Deficiency (GHD) or Turner's syndrome but other indications have also been suggested including long-term treatment of growth failure in children who were born short for gestational age (SGA), for treatment of patients with Prader-Willi syndrome (PWS), chronic renal insufficiency (CRI), Aids wasting, and Aging.

A major biological effect of growth hormone (GH) is to promote growth in young mammals and maintenance of tissues in older mammals. The organ systems affected include the skeleton, connective tissue, muscles, and viscera such as liver, intestine, and kidneys. Growth hormones exert their effect through interaction with specific receptors on the target cell's membrane. hGH is a member of a family of homologous hormones that include placental lactogens, prolactins, and other genetic and species variants or growth hormone (Nicoll, C. S., et al. (1986) Endocrine Reviews 7: 169). hGH is unusual among these in that it exhibits broad species specificity and binds to either the cloned somatogenic (Leung, D. W., et al. [1987] Nature 330; 537) or prolactin receptor (Boutin, J. M., et al. [1988] Cell; 53: 69). The cloned gene for hGH has been expressed in a secreted form in Escherichia coli (Chang, C. N., et al. [1987] Gene 55:189), and its DNA and amino acid sequence has been reported (Goeddel, et al. [1979) Nature 281: 544; Gray, et al. [1985] Gene 39:247).

Human growth hormone (hGH) participates in much of the regulation of normal human growth and development. This pituitary hormone exhibits a multitude of biological effects including linear growth (somatogenesis), lactation, activation of macrophages, insulin-like and diabetogenic effects among others (Chawla, R, K. (1983) Ann. Rev. Med. 34, 519; Edwards, C. K. et al. (1988) Science 239, 769; Thomer, M. O., et al. (1988) J. Clin. Invest. 81:745). Growth hormone deficiency in children leads to dwarfism, which has been successfully treated for more than a decade by exogenous administration of hGH.

Human growth hormone (hGH) is a single-chain polypeptide consisting of 191 amino acids (molecular weight 21,500). Disulfide bonds link positions 53 and 165 and positions 182 and 189. Niall, Nature, New Biology, 230:90 (1971). hGH is a potent anabolic agent, especially due to retention of nitrogen, phosphorus, potassium, and calcium. Treatment of hypophysectomized rats with GH can restore at least a portion of the growth rate of the rats. Moore et al., Endocrinology 122:2920-2926 (1988). Among its most striking effects in hypo pituitary (GH-deficient) subjects is accelerated linear growth of bone-growth-plate-cartilage resulting in increased stature. Kaplan, Growth Disorders in Children and Adolescents (Springfield, Ill.: Charles C. Thomas, 1964).

hGH causes a variety of physiological and metabolic effects in various animal models including linear bone growth, lactation, activation of macrophages, insulin-like and diabetogenic effects, and others (R. K. Chawla et al., Annu. Rev. Med. 34:519 (1983); O. G. P. Isaksson et al., Annu. Rev. Physiol. 47, 483 (1985); C. K. Edwards et al., Science 239, 769 (1988); M. O. Thomer and M. L. Vance, J. Clin. Invest. 82:745 (1988); J. P. Hughes and H. G. Friesen, Ann. Rev. Physiol. 47:469 (1985)). It has been reported that, especially in women after menopause, GH secretion declines with age. Millard et al., Neurobiol. Aging, 11:229-235 (1990); Takahashi et al., Neuroendocrinology M, L6- 137-142 (1987). See also Rudman et al., J. Clin. Invest., 67:1361-1369 (1981) and Blackman, Endocrinology and Aging, 16:981 (1987). Moreover, a report exists that some of the manifestations of aging, including decreased lean body mass, expansion of adipose-tissue mass, and the thinning of the skin, can be reduced by GH treatment three times a week. See, e.g., Rudman et al., N. Eng. J. Med., 323:1-6 (1990) and the accompanying article in the same journal issue by Dr. Vance (pp. 52-54). These biological effects derive from the interaction between hGH and specific cellular receptors. Two different human receptors have been cloned, the hGH liver receptor (D. W. Leung et al., Nature 330:537(1987)) and the human prolactin receptor (J. M. Boutin et al., Mol. Endocrinology. 3:1455 (1989)). However, there are likely to be others including the human placental lactogen receptor (M. Freemark, M. Comer, G. Komer, and S. Handwerger, Endocrinol. 120:1865 (1987)). These homologous receptors contain a glycosylated extracellular hormone binding domain, a single transmembrane domain, and a cytoplasmic domain, which differs considerably in sequence and size. One or more receptors are assumed to play a determining role in the physiological response to hGH.

It is generally observed that physiologically active proteins administered into a body can show their pharmacological activity only for a short period of time due to their high clearance rate in the body. Furthermore, the relative hydrophobicity of these proteins may limit their stability and/or solubility.

For the purpose of decreasing the clearance rate, improving stability or abolishing antigenicity of therapeutic proteins, some methods have been proposed wherein the proteins are chemically modified with water-soluble polymers. Chemical modification of this type may block effectively a proteolytic enzyme from physical contact with the protein backbone itself, thus preventing degradation. Chemical attachment of certain water-soluble polymers may effectively reduce renal clearance due to increased hydrodynamic volume of the molecule. Additional advantages include, under certain circumstances, increasing the stability and circulation time of the therapeutic protein, increasing solubility, and decreasing immunogenicity. Poly(alkylene oxide), notably poly(ethylene glycol) (PEG), is one such chemical moiety that has been used in the preparation of therapeutic protein products (the verb “pegylate” meaning to attach at least one PEG molecule). The attachment of poly(ethylene glycol) has been shown to protect against proteolysis, Sada, et al., J. Fermentation Bioengineering 71: 137-139 (1991), and methods for attachment of certain poly(ethylene glycol) moieties are available. See U.S. Pat. No. 4,179,337, Davis et al., “Non-Immunogenic Polypeptides,” issued Dec. 18, 1979; and U.S. Pat. No. 4,002,531, Royer, “Modifying Enzymes with Polyethylene Glycol and Product Produced Thereby,” issued Jan. 11, 1977. For a review, see Abuchowski et al., in Enzymes as Drugs. (J. S. Holcerberg and J. Roberts, eds. pp. 367-383 (1981)).

Other water-soluble polymers have been used, such as copolymers of ethylene glycol/propylene glycol, carboxymethylcellulose, dextran, poly(vinyl alcohol), poly(vinyl pyrrolidone), poly(-1,3-dioxolane), poly(1,3,6-trioxane), ethylene/maleic anhydride copolymer, poly- amino acids (either homopolymers or random copolymers).

A number of examples of pegylated therapeutic proteins have been described. ADAGEN®, a pegylated formulation of adenosine deaminase, is approved for treating severe combined immunodeficiency disease. ONCASPAR®, a pegylated L-asparaginase has been approved for treating hypersensitive ALL patients. Pegylated superoxide dismutase has been in clinical trials for treating head injury. Pegylated α-interferon (U.S. Pat. Nos. 5,738,846, 5,382,657) has been approved for treating hepatitis; pegylated glucocerebrosidase and pegylated hemoglobin are reported to have been in preclinical testing. Another example is pegylated IL-6, EF 0 442 724, entitled, “Modified hIL-6,” which discloses poly(ethylene glycol) molecules added to IL-6.

Another specific therapeutic protein, which has been chemically modified, is granulocyte colony stimulating factor, (G-CSF). G-CSF induces the rapid proliferation and release of neutrophilic granulocytes to the blood stream, and thereby provides therapeutic effect in fighting infection. European patent publication EP 0 401 384, published Dec. 12, 1990, entitled, “Chemically Modified Granulocyte Colony Stimulating Factor,” describes materials and methods for preparing G-CSF to which poly(ethylene glycol) molecules are attached. Modified G-CSF and analogs thereof are also reported in EP 0 473 268, published Mar. 4, 1992, entitled “Continuous Release Pharmaceutical Compositions Comprising a Polypeptide Covalently Conjugated To A Water Soluble Polymer,” stating the use of various G-CSF and derivatives covalently conjugated to a water soluble particle polymer, such as poly(ethylene glycol). A modified polypeptide having human granulocyte colony stimulating factor activity is reported in EP 0 335 423 published Oct. 4, 1989. Provided in U.S. Pat. No. 5,824,784 are methods for N-terminally modifying proteins or analogs thereof, and resultant compositions, including novel N-terminally chemically modified G-CSF compositions. U.S. Pat. No. 5,824,778 discloses chemically modified G-CSF.

For poly(ethylene glycol), a variety of means have been used to attach the poly(ethylene glycol) molecules to the protein. Generally, poly(ethylene glycol) molecules are connected to the protein via a reactive group found on the protein.

Amino groups, such as those on lysine residues or at the N-terminus, are convenient for such attachment. For example, Royer (U.S. Pat. No. 4,002,531, above) states that reductive alkylation was used for attachment of poly(ethylene glycol) molecules to an enzyme. Chamow et al., Bioconjugate Chem. 5: 133-140 (1994) report the modification of CD4 immunoadhesin with monomethoxypoly(ethylene glycol) aldehyde via reductive alkylation. The authors report that 50% of the CD4-Ig was MePEG-modified under conditions allowing control over the extent of pegylation. Id. at page 137. The authors also report that the in vitro binding capability of the modified CD4-Ig (to the protein gp 120) decreased at a rate correlated to the extent of MePEGylation Ibid. U.S. Pat. No. 4,904,584, Shaw, issued Feb. 27, 1990, relates to the modification of the number of lysine residues in proteins for the attachment of poly(ethylene glycol) molecules via reactive amine groups.

WO 93/00109 relates to a method for stimulating a mammal's or avian's GH responsive tissues comprising, maintaining a continuous, effective plasma GH concentration for a period of 3 or more days. One way of achieving such plasma concentration is stated to be by use of GH coupled to a macromolecular substance such as PEG (polyethylene glycol). The coupling to a macromolecular substance is stated to result in improved half-life. PEGylated human growth hormone has been reported in WO 93/00109 using mPEG aldehyde-5000 and mPEG N-hydroxysuccinmidyl ester(mPEG-NHS-5000). The use of mPEG-NHS resulted in heterogeneous mixtures of multiply PEGylated forms of hGH. WO 93/00109 also discloses the use of mPEG-maleimide to PEGylate cysteine hGH variants.

WO 99/03887 discloses a cysteine variant growth hormone that is PEGylated. Designated as BT-005, this conjugate is purported to be more effective at stimulating weight gain in growth hormone deficient rats and to have a longer half-life than hGH.

PEGylated human growth hormone has also been reported in Clark et al. using succinimidyl ester of carboxymethylated PEG (Journal of Biological Chemistry 271:21969-21977, 1996). Clark et al. describes derivates of hGH of increasing size using mPEG-NHS-5000, which selectively conjugates to primary amines. Increasing levels of PEG modification reduced the affinity for its receptor and increased the EC₅₀ in a cell-based assay up to 1500 fold. Olson et al., Polymer Preprints 38:568-569, 1997 discloses the use of N-hydroxysuccinimide (NHS)PEG and succinimidyl propionate (SPA)PEG to achieve multiply PEGylated hGH species.

WO 94/20069 prophetically discloses PEGylated hGH as part of a formulation for pulmonary delivery.

U.S. Pat. No. 4,179,337 discloses methods of PEGylating enzymes and hormones to obtain physiologically active non-immunogenic, water-soluble polypeptide conjugates. GH is mentioned as one example of a hormone to be PEGylated.

EP 458064 A2 discloses PEGylation of introduced or naturally present cysteine residues in somatotropin. EP 458064 A2 further mentions the incorporation of two cysteine residues in a loop termed the omega loop stated to be located at residues 102-112 in wild type bovine somatotropin, more specifically EP 458064 A2 discloses the substitution of residues numbered 102 and 112 of bovine somatotropin from Ser to Cys and Tyr to Cys, respectively.

WO 95/11987 suggests attachment of PEG to the thio group of a cysteine residue being either present in the parent molecule or introduced by site directed mutagenesis. WO 95/11987 relates to PEGylation of protease nexin-1, however PEGylation in general of hGH and other proteins is suggested as well.

WO 99/03887 discloses, e.g., growth hormone modified by insertion of additional cys 25 serine residues and attachment of PEG to the introduced cysteine residues.

WO 00/42175 relates to a method for making proteins containing free cysteine residues for attachment of PEG. WO 00/42175 discloses the following muteins of hGH: T3C, S144C and T148C and the cysteine PEGylation thereof.

WO 97/11178 (as well as U.S. Pat. No. 5,849,535, U.S. Pat. No. 6,004,931, and U.S. Pat. No. 6,022,711) relates to the use of GH variants as agonists or antagonists of hGH. WO 97/11178 also discloses PEGylation of hGH, including lysine PEGylation and the introduction or replacement of lysine (e.g. K168A and K172R). WO 9711178 also discloses the substitution G120K.

Wo 03/044056 discloses a variety of PEGylated hGH species including a branched 40K PEG aldehyde hGH conjugate.

The previous reports of PEGylated hGH require the attachment of multiple PEGs, which results in undesirable product heterogeneity, to achieve a hydrodynamic volume greater than the 70K molecular weight cut-off of the kidney filtration as described (Knauf, M. J. et al, J. Biol. Chem. 263:15064-15070,1988).

Currently administration of rhGH is daily for a long period of time, and therefore a less frequent administration would be highly desirable. A hGH molecule with a longer circulation half-life would decrease the number of necessary administrations and potentially provide more optimal therapeutic hGH levels with concomitant enhanced therapeutic effect.

Despite a number of attempts to PEGylate hGH, there is still an unmeet need for a PEGylated hGH molecule with the appropriate properties to be a viable commercial product. The present invention provides PEG-hGH conjugates having a single PEG attached predominately at the N-terminal phenylalanine of hGH, which provides advantages over other PEG-hGH conjugates. The attachment of multiple low molecular weight (5 Kd) PEGs at α- or β-amino sites (N-terminus and nine lysines in hGH) using mPEG aldehyde-5000 or mPEG N-hydroxysuccinmidyl ester (mPEG-NHS-5000) has been described in WO 93/00109, Clark et al. (Journal of Biological Chemistry 271:21969-21977, 1996, and Olson et al. (Polymer Preprints 38:568-569, 1997). This results in a heterogeneous population. As an illustration hGH with nine lysines may have some molecules having ten PEGs attached, some with nine, some with eight, some with seven, some with six, some with five, some with four, some with three, some with two, some with one and some with zero. And, among the molecules with several, the PEG may not be attached at the same location on different molecules. This resulting heterogeneity is disadvantageous when developing a therapeutic product making conjugation, purification, and characterization difficult, costly, and highly irreproducible. Another approach (WO 00/42175) has been to use hGH variants containing free cysteine residues for attachment of PEG. However, this approach can lead to incorrectly folded protein having incorrectly paired disulfide bonds and resulting in a heterogeneous PEGylated product that has the PEG attached at some or all of the cysteines. Having multiple PEGs attached to multiple sites may lead to molecules that have less stable bounds between the PEG and the various sites, which can become dissociated at different rates. This makes it difficult to accurately predict the pharmacokinetics of the product resulting in inaccurate dosing. A heterogeneous product also posses unwanted problems in obtaining regulatory approval for the therapeutic product.

Therefore, it would be desirable to have a PEGylated hGH molecule that has a single PEG attached at a single site. The present invention addresses this need in a number of ways.

### SUMMARY OF THE INVENTION

The present invention relates to chemically modified hGH and agonist variants thereof, which have at least one improved chemical or physiological property selected from but not limited to decreased clearance rate, increased plasma residency duration, increased stability, improved solubility, and decreased antigenicity. Thus, as described below in more detail, the present invention has a number of aspects relating to chemically modifying polypeptides including but not limited to hGH and agonist variants thereof as well as specific modifications using a poly(ethylene glycol) butyraldehyde moiety.

The present invention also relates to methods of producing the chemically modified hGH and agonist variants thereof. Particularly, the present invent relates to a method of producing a chemically modified hGH using butyraldehyde, which results in greater N-terminal selectivity of attachment.

The present invention also relates to compositions comprising the chemically modified hGH and agonist variants thereof.

The modified hGH and agonist variants thereof of the present invention may be useful in the treatment of, but not limited to, dwarfism (GHD), Adult GHD, Turner's syndrome, long-term treatment of growth failure in children who were born short for gestational age (SGA), for treatment of patients with Prader-Willi syndrome (PWS), chronic renal insufficiency (CRI), Aids wasting, Aging, End-stage Renal Failure, and Cystic Fibrosis.

### BRIEF DESCRIPTION OF THE DRAWINGS

FIG. 1 is a HPLC tracing of tryptic map analysis of the reaction of hGH and 40K branched butyrylaldehyde hGH or 40K branched aldehyde hGH. The top panel is the tryptic map of 40K Branched butyraldehyde hGH. The middle panel is the tryptic map of 40K Branched aldehyde hGH. The bottom panel is the tryptic map of unPEGylated hGH. T1 is the N-terminal tryptic fragment.

FIG. 2 shows the amino acid sequence of human growth hormone (SEQ ID NO:1).

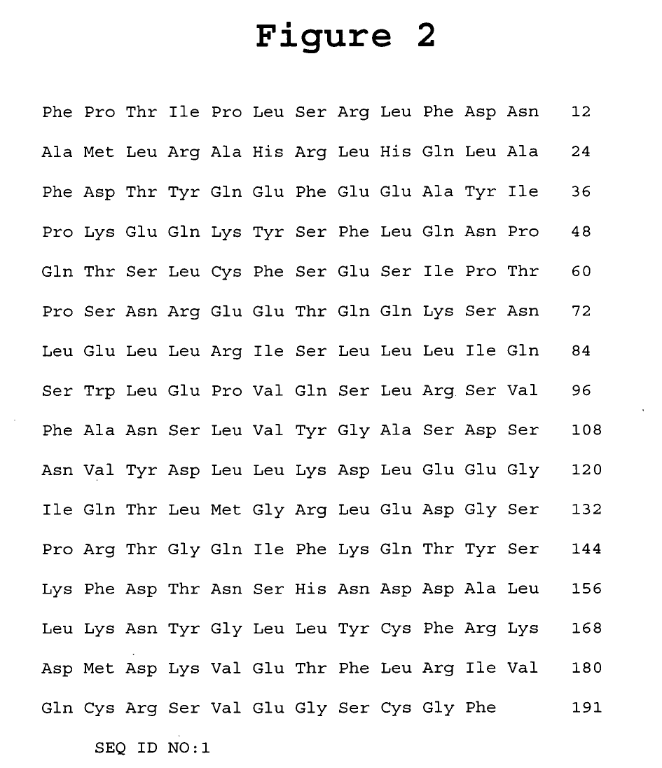

FIG. 3 shows 40K Branched butyraldehyde hGH efficacy in Rat Weight Gain Assay. Hypophysectomized female Sprague-Dawley rats were purchased at the age of 4-5 weeks (100-125 g) from Harlan Labs. Upon entering the animal facilities, the animals were maintained at a constant room temperature of 80° F. and weighed daily for 4-10 days in order to establish basal growth rates. Starting at day 0, rats (˜100 g) in control groups then received one daily subcutaneous injection of ˜0.3 mg/kg hGH (solid circles), or PBS (open circles), for eleven consecutive days. The 40K Branched butyraldehyde hGH test group (solid squares) received single doses of 1.8 mg/kg of PHA-794428 on days 0 and 6. There were 8-10 animals per group. Average growth +/−SEM is plotted.

FIG. 4. shows the Dose-Responsive Growth Promoting Effects of 40K Branched butyraldehyde hGH in Rats. This efficacy study was performed in a manner similar to that described in FIG. 3 except that a varied single dose of 40K Branched butyraldehyde hGH was administered (day 0, only) and the study ran for 6 days. Control groups received once-daily injections of either 0.3 mg/kg hGH (solid circles), or PBS vehicle (open circles) for six consecutive days. 40K Branched butyraldehyde hGH was dosed at 1.8 mg/kg (solid squares), 0.6 mg/kg (open squares), 0.2 mg/kg (solid triangles) or 0.067 mg/kg (open triangles). There were 8 animals per group.

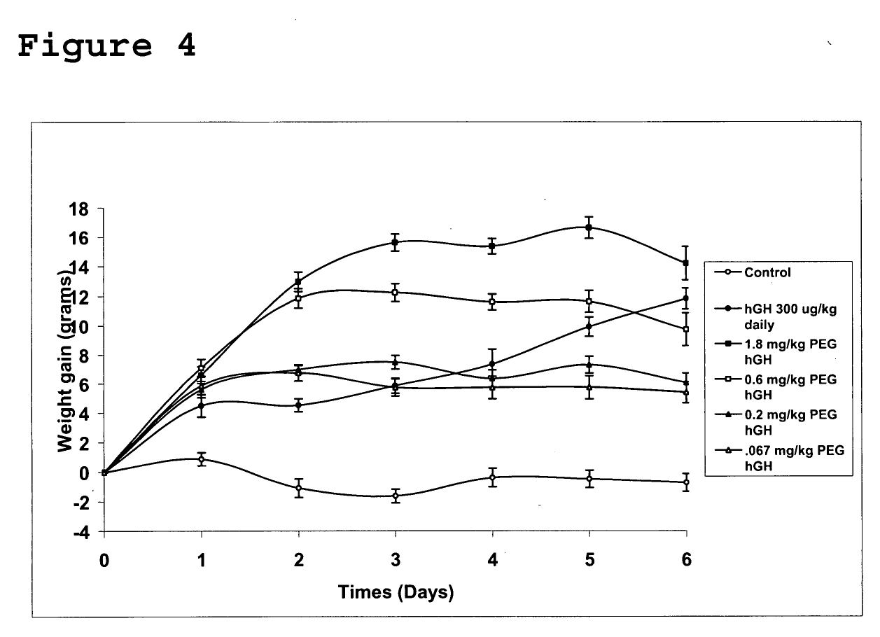

FIG. 5 shows tibial growth in response to 40K Branched butyraldehyde hGH. Hypophysectomized rats were treated as described in FIG. 3. At day 11 animals were sacrificed, left tibias were removed and X-rayed and bone lengths were measured using a caliper. Average length +/−SEM is plotted. Asterisks denote significant differences from control group (p<0.05).

FIG. 6 shows plasma IGF-1 levels for six-day efficacy study. Animals were treated as described FIG. 4. Blood samples were taken at the various times and the serum IGF-1 levels determined by ELISA. Plotted are averages +/−SEM.

### DETAILED DESCRIPTION

hGH and agonist variants thereof are members of a family of recombinant proteins, described in U.S. Pat. No. 4,658,021 (methionyl human growth hormone—Met-1-191 hGH) and U.S. Pat. No. 5,633,352. Their recombinant production and methods of use are detailed in U.S. Pat. No. 4,342,832, U.S. Pat. No. 4,601,980; U.S. Pat. No. 4,898,830; U.S. Pat. No. 5,424,199; and U.S. Pat. No. 5,795,745.

Any purified and isolated hGH or agonist variant thereof, which is produced by host cells such as E. coli and animal cells transformed or transfected by using recombinant genetic techniques, may be used in the present invention. Additional hGH variants are described in U.S. Pat. No. 6,143,523 and WO 92/09690 published Jun. 11, 1992. Among them, hGH or agonist variant thereof, which is produced by the transformed E. coli, is particularly preferable. Such hGH or agonist variant thereof may be obtained in large quantities with high purity and homogeneity. For example, the above hGH or agonist variant thereof may be prepared according to a method disclosed in U.S. Pat. No. 4,342,832, U.S. Pat. No. 4,601,980; U.S. Pat. No. 4,898,830; U.S. Pat. No. 5,424,199; and U.S. Pat. No. 5,795,745. The term “substantially has the following amino acid sequence” means that the above amino acid sequence may include one or more amino-acid changes (deletion, addition, insertion or replacement) as long as such changes will not cause any disadvantageous non-similarity in function to hGH or agonist variant thereof. It is more preferable to use the hGH or agonist variant thereof substantially having an amino acid sequence, in which at least one lysine, aspartic acid, glutamic acid, unpaired cysteine residue, a free N-terminal α-amino group or a free C-terminal carboxyl group, is included.

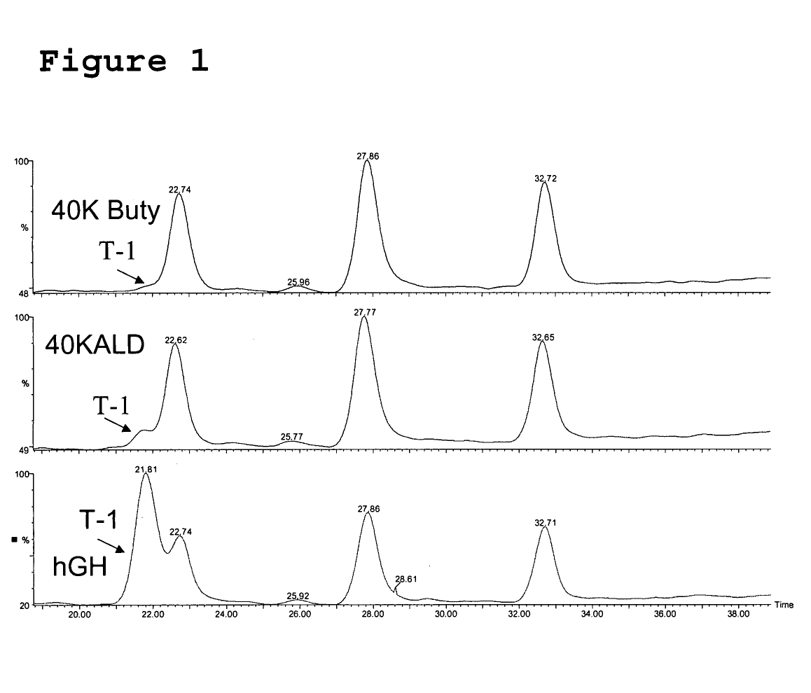

According to the present invention, poly(ethylene glycol) is covalently bound through amino acid residues of hGH or agonist variant thereof. A variety of activated poly(ethylene glycol)s having a number of different functional groups, linkers, configurations, and molecular weights are known to one skilled in the art, which may be used to create PEG-hGH conjugates or PEG-hGH agonist variant conjugates (for reviews see Roberts M. J. et al., Adv. Drug Del. Rev. 54:459-476, 2002), Harris J. M. et al., Drug Delivery Sytems 40:538-551, 2001) The present invention relates to a method of using aldehyde chemistry to direct selectivity of the PEG moiety to the N-terminus using a butyrylaldehyde linker moiety. The butyrylaldehyde linker results in increased N-terminal specificity compared to acetaldehyde linker (Table 1 and FIG. 1).

An embodiment of the present invention is a human growth hormone-PEG conjugate having the structure of Formula I or Formula II

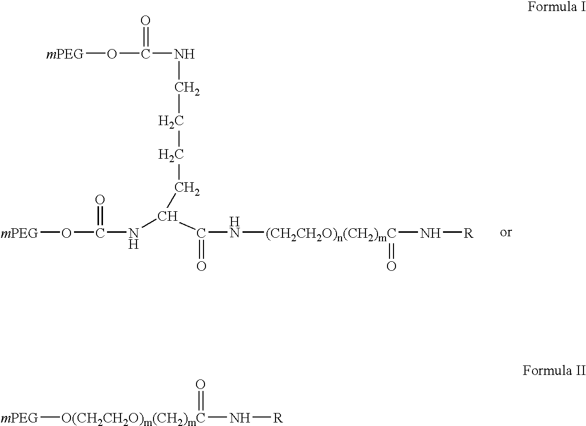

wherein

n is an integer between 1 and 10;

m is an integer between 1 and 10;

R is human growth hormone, methionyl growth hormone or a human growth hormone variant.

A specific embodiment is a human growth hormone-PEG conjugate having the structure:

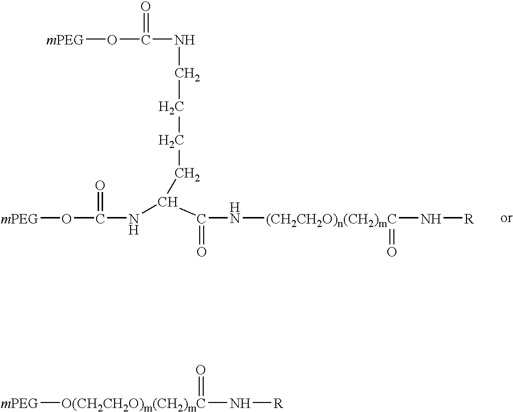

wherein R is human growth hormone, methionyl human growth hormone or a human growth hormone variant.

Another specific embodiment of the present invention human growth hormone-PEG conjugate wherein the human growth hormone has the amino acid sequence of SEQ ID NO:1.

A specific embodiment of the present invention is a human growth hormone-PEG conjugate wherein greater than 80%, more preferably 81%, more preferably 82%, more preferably 83%, more preferably 84%, more preferably 85%, more preferably 86%, more preferably 87%, more preferably 88%, more preferably 89%, more preferably 90%, more preferably 91%, more preferably 92%, more preferably 93%, more preferably 94%, more preferably 95%, more preferably 96%, more preferably 97, and more preferably 98% of the polyethylene glycol is conjugated to the amino-terminal phenylalanine of the amino acid sequence of SEQ ID NO:1.

Another specific embodiment of the present invention is a human growth hormone-PEG conjugate wherein greater than 90% of the polyethylene glycol is conjugated to the amino-terminal phenylalanine of the amino acid sequence of SEQ ID NO:1.

Another specific embodiment of the present invention is a human growth hormone-PEG conjugate wherein greater than 95% of the polyethylene glycol is conjugated to the amino-terminal phenylalanine of the amino acid sequence of SEQ ID NO:1.

Another specific embodiment of the present invention is a human growth hormone-PEG conjugate wherein greater than 98% of the polyethylene glycol is conjugated to an amino-terminal phenylalanine of the amino acid sequence of SEQ ID NO:1.

The poly(ethylene glycol) used in the present invention is not restricted to any particular form or molecular weight range. The poly(ethylene glycol) molecular weight may between about 500 and about 100,000 Dalton. The term “about” indicating that in preparations of polyethylene glycol, some molecules will weigh more, some less, than the stated molecular weight and the stated molecular weight refers to the average molecular weight. It is understood that there is some degree of polydispersity associated with polymers such as poly(ethylene glycol). It is preferable to use PEGs with low polydispersity. Normally, a PEG with molecular weight of about 500 to about 60,000 is used. A specific PEG molecular weight range of the present invention is from about 1,000 to about 40,000. In another specific embodiment the PEG molecular weight is greater than about 5,000 to about 40,000. In another specific embodiment the PEG molecular weight about 20,000 to about 40,000. Other sizes may be used, depending on the desired therapeutic profile (e.g. duration of sustained release desired, the effects, if any on biological activity, the degree or lack of antigenicity and other known effects of the polyethylene to a therapeutic protein. For example the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 Dalton.

In another embodiment the poly(ethylene glycol) is a branched PEG having more than one PEG moiety attached (see U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660; Kodera Y., Bioconjugate Chemistry 5:283-288 (1994); and WO 02/09766. In a preferred embodiment the molecular weight of each poly(ethylene glycol) of the branched PEG is about 5,000-20,000. In a specific embodiment the molecular weight of each poly(ethylene glycol) of the branched PEG is about 20,000.

Poly(alkylene oxide)s, notably poly(ethylene glycol)s, are bound to hGH or agonist variant thereof via a terminal reactive group, which may or may not leave a linking moiety (spacer) between the PEG and the protein. In order to form the hGH conjugates or agonist variant thereof of the present invention, polymers such as poly(alkylene oxide) are converted into activated forms, as such term is known to those of ordinary skill in the art. The reactive group, for example, is a terminal reactive group, which mediates a bond between chemical moieties on the protein and poly(ethylene glycol). Typically, one or both of the terminal polymer hydroxyl end-groups, (i.e. the alpha and omega terminal hydroxyl groups) are converted into reactive functional groups, which allows covalent conjugation. This process is frequently referred to as “activation” and the poly(ethylene glycol) product having the reactive group is hereinafter referred to as “an activated poly(ethylene glycol)”. In a specific embodiment one of the terminal polymer hydroxyl end-groups is converted or capped with a non-reactive group. In a specific embodiment one of the terminal polymer hydroxyl end-groups is converted or capped with a methyl group. As used herein, the term “mPEG” refers to a PEG, which is capped at one end with a methyl group. The mPEG can be represented structurally as

CH₃O—(CH₂CH₂O)ₙ—H

Polymers containing both α and ε linking groups are referred to as “bis-activated poly(alkylene oxides)” and are referred to as “bifunctional”. Polymers containing the same reactive group on α and ε terminal hydroxyls are sometimes referred to as “homobifunctional” or “homobis-activated”. Polymers containing different reactive groups on α and ε terminal hydroxyls are sometimes referred to as “heterobifunctional” (see for example WO 01/26692) or “heterobis-activated”. Polymers containing a single reactive group are referred to as “mono-activated” polyalkylene oxides or “mono-functional”. Other substantially non-antigenic polymers are similarly “activated” or “functionalized”.

The activated polymers are thus suitable for mediating a bond between chemical moieties on the protein, such as α- or ε-amino, carboxyl or thiol groups, and poly(ethylene glycol). Bis-activated polymers can react in this manner with two protein molecules or one protein molecule and a reactive small molecule in another embodiment to effectively form protein polymers or protein-small molecule conjugates through cross linkages.

In one preferred embodiment of the invention secondary amine or amide linkages are formed using the N-terminal α-amino group or ε-amino groups of lysine of hGH or agonist variant thereof and the activated PEG. In another preferred aspect of the invention, a secondary amine linkage is formed between the N-terminal primary α- or ε-amino group of hGH or agonist variant thereof and single or branched chain PEG aldehyde by reductive alkylation with a suitable reducing agent such as NaCNBH₃, NaBH₃, Pyridine Borane etc. as described in Chamow et al., Bioconjugate Chem. 5: 133-140 (1994), U.S. Pat No. 4,002,531, WO 90/05534, and U.S. Pat. No 5,824,784.

In a preferred embodiment at least 70%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, and most preferably at least 98% of the poly(ethylene glycol) is on the amino terminal α-amino group.

Conjugation reactions, referred to as pegylation reactions, were historically carried out in solution with molar excess of polymer and without regard to where the polymer will attach to the protein. Such general techniques, however, have typically been proven inadequate for conjugating bioactive proteins to non-antigenic polymers while retaining sufficient bioactivity. One way to maintain the hGH or agonist variant thereof bioactivity is to substantially avoid the conjugation of those hGH or agonist variant thereof reactive groups associated with the receptor binding site(s) in the polymer coupling process. Another aspect of the present invention is to provide a process of conjugating poly(ethylene glycol) to hGH or agonist variant thereof maintaining high levels of retained activity.

The chemical modification through a covalent bond may be performed under any suitable condition generally adopted in a reaction of a biologically active substance with the activated poly(ethylene glycol). The conjugation reaction is carried out under relatively mild conditions to avoid inactivating the hGH or agonist variant thereof. Mild conditions include maintaining the pH of the reaction solution in the range of 3 to 10 and the reaction temperatures within the range of from about 0°-37° C. In the cases where the reactive amino acid residues in hGH or agonist variant thereof have free amino groups, the above modification is preferably carried out in a non-limiting list of suitable buffers (pH 3 to 10), including phosphate, MES, citrate, acetate, succinate or HEPES, for 1-48 hrs at 4°-37° C. In targeting N-terminal amino groups with reagents such as PEG aldehydes pH 4-7 is preferably maintained. The activated poly(ethylene glycol) may be used in about 0.01-100 times, preferably about 0.01-2.5 times, the molar amount of the number of free amino groups of hGH or agonist variant thereof. On the other hand, where reactive amino acid residues in hGH or agonist variant thereof have the free carboxyl groups, the above modification is preferably carried out in pH from about 3.5 to about 5.5, for example, the modification with poly(oxyethylenediamine) is carried out in the presence of carbodiimide (pH 4-5) for 1-24 hrs at 4°-37° C. The activated poly(ethylene glycol) may be used in 0.01-300 times the molar amount of the number of free carboxyl groups of hGH or agonist variant thereof.

In separate embodiments, the upper limit for the amount of polymer included in the conjugation reactions exceeds about 1:1 to the extent that it is possible to react the activated polymer and hGH or agonist variant thereof without forming a substantial amount of high molecular weight species, i.e. more than about 20% of the conjugates containing more than about one strand of polymer per molecule of hGH or agonist variant thereof. For example, it is contemplated in this aspect of the invention that ratios of up to about 6:1 can be employed to form significant amounts of the desired conjugates which can thereafter be isolated from any high molecular weight species.

In another aspect of this invention, bifunctionally activated PEG derivatives may be used to generate polymeric hGH or agonist variant thereof-PEG molecules in which multiple hGH or agonist variant thereof molecules are crosslinked via PEG. Although the reaction conditions described herein can result in significant amounts of unmodified hGH or agonist variant thereof, the unmodified hGH or agonist variant thereof can be readily recycled into future batches for additional conjugation reactions. The processes of the present invention generate surprisingly very little, i.e. less than about 30% and more preferably, less than about 10%, of high molecular weight species and species containing more than one polymer strand per hGH or agonist variant thereof. These reaction conditions are to be contrasted with those typically used for polymeric conjugation reactions wherein the activated polymer is present in several-fold molar excesses with respect to the target. In other aspects of the invention, the polymer is present in amounts of from about 0.1 to about 50 equivalents per equivalent of hGH or agonist variant thereof. In other aspects of the invention, the polymer is present in amounts of from about 1 to about 10 equivalents per equivalent of hGH or agonist variant thereof.

The conjugation reactions of the present invention initially provide a reaction mixture or pool containing mono- and di-PEG-hGH conjugates, unreacted hGH, unreacted polymer, and usually less than about 20% high molecular weight species. The high molecular weight species include conjugates containing more than one polymer strand and/or polymerized PEG-hGH or agonist variant thereof species. After the unreacted species and high molecular weight species have been removed, compositions containing primarily mono- and di-polymer-hGH or agonist variant thereof conjugates are recovered. Given the fact that the conjugates for the most part include a single polymer strand, the conjugates are substantially homogeneous. These modified hGH or agonist variant thereof have at least about 0.1% of the in vitro biological activity associated with the native or unmodified hGH or agonist variant thereof as measured using standard FDC-P1 cell proliferation assays, (Clark et al. Journal of Biological Chemistry 271:21969-21977, 1996), receptor binding assay (U.S. Pat. No. 5,057,417), or hypophysectomized rat growth (Clark et al. Journal of Biological Chemistry 271:21969-21977, 1996). In preferred aspects of the invention, however, the modified hGH or agonist variant thereof have about 25% of the in vitro biological activity, more preferably, the modified hGH or agonist variant thereof have about 50% of the in vitro biological activity, more preferably, the modified hGH or agonist variant thereof have about 75% of the in vitro biological activity, and most preferably the modified hGH or agonist variant thereof have equivalent or improved in vitro biological activity.

The processes of the present invention preferably include rather limited ratios of polymer to hGH or agonist variant thereof. Thus, the hGH or agonist variant thereof conjugates have been found to be predominantly limited to species containing only one strand of polymer. Furthermore, the attachment of the polymer to the hGH or agonist variant thereof reactive groups is substantially less random than when higher molar excesses of polymer linker are used. The unmodified hGH or agonist variant thereof present in the reaction pool, after the conjugation reaction has been quenched, can be recycled into future reactions using ion exchange or size exclusion chromatography or similar separation techniques.

A poly(ethylene glycol)-modified hGH or agonist variant thereof, namely chemically modified protein according to the present invention, may be purified from a reaction mixture by conventional methods which are used for purification of proteins, such as dialysis, salting-out, ultrafiltration, ion-exchange chromatography, hydrophobic interaction chromatography (HIC), gel chromatography and electrophoresis. Ion-exchange chromatography is particularly effective in removing unreacted poly(ethylene glycol) and hGH or agonist variant thereof. In a further embodiment of the invention, the mono- and di-polymer-hGH or agonist variant thereof species are isolated from the reaction mixture to remove high molecular weight species, and unmodified hGH or agonist variant thereof. Separation is effected by placing the mixed species in a buffer solution containing from about 0.5-10 mg/mL of the hGH or agonist variant thereof-polymer conjugates. Suitable solutions have a pH from about 4 to about 10. The solutions preferably contain one or more buffer salts selected from KCl, NaCl, K₂HPO₄, KH₂PO₄, Na₂HPO₄, NaH₂PO₄, NaHCO₃, NaBO₄, CH₃CO₂H, and NaOH.

Depending upon the reaction buffer, the hGH or agonist variant thereof polymer conjugate solution may first have to undergo buffer exchange/ultrafiltration to remove any unreacted polymer. For example, the PEG-hGH or agonist variant thereof conjugate solution can be ultrafiltered across a low molecular weight cut-off (10,000 to 30,000 Dalton) membrane to remove most unwanted materials such as unreacted polymer, surfactants, if present, or the like.

The fractionation of the conjugates into a pool containing the desired species is preferably carried out using an ion exchange chromatography medium. Such media are capable of selectively binding PEG-hGH or agonist variant thereof conjugates via differences in charge, which vary in a somewhat predictable fashion. For example, the surface charge of hGH or agonist variant thereof is determined by the number of available charged groups on the surface of the protein. These charged groups typically serve as the point of potential attachment of poly(alkylene oxide) polymers. Therefore, hGH or agonist variant thereof conjugates will have a different charge from the other species to allow selective isolation.

Strongly polar anion or cation exchange resins such as quaternary amine or sulfopropyl resins, respectively, are used for the method of the present invention. Ion exchange resins are especially preferred. A non-limiting list of included commercially available cation exchange resins suitable for use with the present invention are SP-hitrap®, SP Sepharose HP® and SP Sepharose® fast flow. Other suitable cation exchange resins e.g. S and CM resins can also be used. A non-limiting list of anion exchange resins, including commercially available anion exchange resins, suitable for use with the present invention are Q-hitrap®, Q Sepharose HP®, and Q sepharose® fast flow. Other suitable anion exchange resins, e.g. DEAE resins, can also be used.

For example, the anion or cation exchange resin is preferably packed in a column and equilibrated by conventional means. A buffer having the same pH and osmolality as the polymer conjugated hGH or agonist variant thereof solution is used. The elution buffer preferably contains one or more salts selected from KCl, NaCl, K₂HPO₄, KH₂PO₄, Na₂HPO₄, NaH₂PO₄, NaHCO₃, NaBO₄, and (NH₄)₂CO₃. The conjugate-containing solution is then adsorbed onto the column with unreacted polymer and some high molecular weight species not being retained. At the completion of the loading, a gradient flow of an elution buffer with increasing salt concentrations is applied to the column to elute the desired fraction of polyalkylene oxide-conjugated hGH or agonist variant thereof. The eluted pooled fractions are preferably limited to uniform polymer conjugates after the cation or anion exchange separation step. Any unconjugated hGH or agonist variant thereof species can then be back washed from the column by conventional techniques. If desired, mono and multiply pegylated hGH or agonist variant thereof species can be further separated from each other via additional ion exchange chromatography or size exclusion chromatography.

Techniques utilizing multiple isocratic steps of increasing concentration of salt or pH can also be used. Multiple isocratic elution steps of increasing concentration will result in the sequential elution of di- and then mono-hGH or agonist variant thereof-polymer conjugates.

The temperature range for elution is between about 4° C. and about 25° C. Preferably, elution is carried out at a temperature of from about 4° C. to about 22° C. For example, the elution of the PEG-hGH or agonist variant thereof fraction is detected by UV absorbance at 280 nm. Fraction collection may be achieved through simple time elution profiles.

A surfactant can be used in the processes of conjugating the poly(ethylene glycol) polymer with the hGH or agonist variant thereof moiety. Suitable surfactants include ionic-type agents such as sodium dodecyl sulfate (SDS). Other ionic surfactants such as lithium dodecyl sulfate, quaternary ammonium compounds, taurocholic acid, caprylic acid, decane sulfonic acid, etc. can also be used. Non-ionic surfactants can also be used. For example, materials such as poly(oxyethylene) sorbitans (Tweens), poly(oxyethylene) ethers (Tritons) can be used. See also Neugebauer, A Guide to the Properties and Uses of Detergents in Biology and Biochemistry (1992) Calbiochem Corp. The only limitations on the surfactants used in the processes of the invention are that they are used under conditions and at concentrations that do not cause substantial irreversible denaturation of the hGH or agonist variant thereof and do not completely inhibit polymer conjugation. The surfactants are present in the reaction mixtures in amounts from about 0.01-0.5%; preferably from 0.05-0.5%; and most preferably from about 0.075-0.25%. Mixtures of the surfactants are also contemplated.

It is thought that the surfactants provide a temporary, reversible protecting system during the polymer conjugation process. Surfactants have been shown to be effective in selectively discouraging polymer conjugation while allowing lysine-based or amino terminal-based conjugation to proceed.

The present poly(ethylene glycol)-modified hGH or agonist variant thereof has a more enduring pharmacological effect, which may be possibly attributed to its prolonged half-life in vivo.

Furthermore, it is observed that the present poly(ethylene glycol)-modified hGH or agonist variant thereof may be useful for the treatment of hypo pituitary dwarfism (GHD), Turner's syndrome, growth failure in children who were born short for gestational age (SGA), Prader-Willi syndrome (PWS), chronic renal insufficiency (CRI), Aids wasting, and Aging.

The present poly(ethylene glycol)-modified hGH or agonist variant thereof may be formulated into pharmaceuticals containing also a pharmaceutically acceptable diluent, an agent for preparing an isotonic solution, a pH-conditioner and the like in order to administer them into a patient.

The above pharmaceuticals may be administered subcutaneously, intramuscularly, intravenously, pulmonary, intradermally, or orally, depending on a purpose of treatment. A dose may be also based on the kind and condition of the disorder of a patient to be treated, being normally between 0.1 mg and 5 mg by injection and between 0.1 mg and 50 mg in an oral administration for an adult

The polymeric substances included are also preferably water-soluble at room temperature. A non-limiting list of such polymers include poly(alkylene oxide) homopolymers such as poly(ethylene glycol) or poly(propylene glycols), poly(oxyethylenated polyols), copolymers thereof and block copolymers thereof, provided that the water solubility of the block copolymers is maintained.

As an alternative to PEG-based polymers, effectively non-antigenic materials such as dextran, poly(vinyl pyrrolidones), poly(acrylamides), poly(vinyl alcohols), carbohydrate-based polymers, and the like can be used. Indeed, the activation of α- and ε-terminal groups of these polymeric substances can be effected in fashions similar to that used to convert poly(alkylene oxides) and thus will be apparent to those of ordinary skill. Those of ordinary skill in the art will realize that the foregoing list is merely illustrative and that all polymer materials having the qualities described herein are contemplated. For purposes of the present invention, “effectively non-antigenic” means all materials understood in the art as being nontoxic and not eliciting an appreciable immunogenic response in mammals.

Definitions

The following is a list of abbreviations and the corresponding meanings as used interchangeably herein:

| | |
|---|---|
| g | gram(s) |
| mg | milligram(s) |
| ml or mL | milliliter(s) |
| RT | room temperature |
| PEG | poly (ethylene glycol) |

The complete content of all publications, patents, and patent applications cited in this disclosure are herein incorporated by reference as if each individual publication, patent, or patent application were specifically and individually indicated to be incorporated by reference.

Although the foregoing invention has been described in some detail by way of illustration and example for the purposes of clarity of understanding, it will be readily apparent to one skilled in the art in light of the teachings of this invention that changes and modifications can be made without departing from the spirit and scope of the present invention. The following examples are provided for exemplification purposes only and are not intended to limit the scope of the invention, which has been described in broad terms above.

In the following examples, the hGH is that of SEQ ID NO:1. It is understood that other members of the hGH or agonist variant thereof family of polypeptides could also be pegylated in a similar manner as exemplified in the subsequent examples.

### EXAMPLES

### Example 1

Branched 40,000 MW PEG-butyrylaldhyde hGH

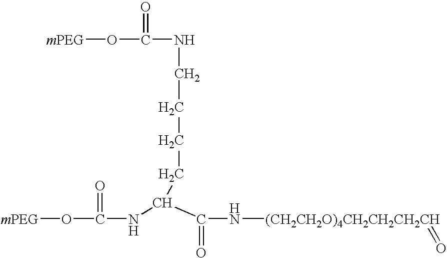

This example demonstrates a method for generation of substantially homogeneous preparations of N-terminally monopegylated hGH by reductive alkylation. Methoxy-branched PEG-butyrylaldehyde reagent of approximately 40,000 MW (Shearwater Corp.) was selectively coupled via reductive amination to the N-terminus of hGH by taking advantage of the difference in the relative pKₐ value of the primary amine at the N-terminus versus pKₐ values of primary amines at the ε-amino position of lysine residues. hGH protein dissolved at 10 mg/mL in 25 mM Hepes (Sigma Chemical, St. Louis, Mo.) pH 7.0, (optionally 25 mM MES (Sigma Chemical , St. Louis, Mo.) pH 6.0, 10 mM Sodium Acetate (Sigma Chemical , St. Louis, Mo.) pH 4.5), was reacted with Methoxy-PEG- butyrylaldehyde, M-PEG-ALD, (Shearwater Corp., Huntsville, Ala.) by addition of M-PEG-ALD to yield a relative PEG:hGH molar ratio of 2:1. Reactions were catalyzed by addition of stock 1M NaCNBH₄ (Sigma Chemical , St. Louis, Mo.), dissolved in H₂O, to a final concentration of 10-50 mM. Reactions were carried out in the dark at RT for 18-24 hours. Reactions were stopped by addition of 1 M Tris (Sigma Chemical, St. Louis, Mo.) ˜pH 7.6 to a final Tris concentration of 50 mM or diluted into appropriate buffer for immediate purification.

Table 1 shows the percent, as determined by Size Exclusion Chromatography, of multi-PEGylated species, mono-PEGylated conjugate, un-reacted PEG, and final purification yield for 40K branched PEG-aldehyde and 40K branched PEG-butyrylaldehyde. The PEG-butyrylalehyde results in increased mono-PEGylated conjugate, decreased levels of un-reacted PEG, and increased final yield compared to PEG-aldehyde.

TABLE 1
Comparison of 40K Branched PEG-ALD-hGH and
40K branched PEG-Butyrylaldehyde-hGH

| Species in the reaction | 40K PEG-aldehyde | 40K PEG- |
|---|---|---|
| mix: | hGH | butyrylaldehyde-hGH |
| multi-PEG product | 4.02% | 5.03% |
| mono-PEG product | 48.70% | 61.02% |
| un-reacted hGH | 41.80% | 29.20% |
| Final purification yield | 30.80% | 44.70% |

### Example 2

Straight Chain 30,000 MW PEG-butyrylaldehyde hGH

Methoxy-linear 30,000 MW PEG-butyrylaldehyde reagent is coupled to the N-terminus of hGH using the procedure described for Example 1.

### Example 3

Straight chain 20,000 MW PEG-butyrylaldehyde hGH

Methoxy-linear 20,000 MW PEG-butyrylaldehyde reagent is coupled to the N-terminus of hGH using the procedure described for Example 1.

### Example 4

Purification of Pegylated hGH

Pegylated hGH species were purified from the reaction mixture to >95% (SEC analysis) using a single ion exchange chromatography step.

Anion Exchange Chromatography

The PEG hGH species were purified from the reaction mixture to >95% (SEC analysis) using a single anion exchange chromatography step. Mono-pegylated hGH was purified from unmodified hGH and multi-pegylated hGH species using anion exchange chromatography. A typical 20K butyrylaldehyde hGH reaction mixture (5-100 mg protein), as described above, was purified on a Q-Sepharose Hitrap column (1 or 5 mL)(Amersham Pharmacia Biotech, Piscataway, N.J.) or Q-Sepharose fast flow column (26/20, 70 mL bed volume)(Amersham Pharmacia Biotech, Piscataway, N.J.) equilibrated in 25 mM HEPES, pH 7.3 (Buffer A). The reaction mixture was diluted 5-10× with buffer A and loaded onto the column at a flow rate of 2.5 mL/min. The column was washed with 8 column volumes of buffer A. Subsequently, the various hGH species were eluted from the column in 80-100 column volumes of Buffer A and a linear NaCl gradient of 0-100 mM. The eluant was monitored by absorbance at 280 nm (A₂₈₀) and 5 mL fractions were collected. Fractions were pooled as to extent of pegylation, e.g., mono, di, tri etc. (as assessed in example 15). The pool was then concentrated to 0.5-5 mg/mL in a Centriprep YM10 concentrator (Amicon, Technology Corporation, Northborough, Mass.). Protein concentration of pool was determined by A₂₈₀ using an extinction coefficient of 0.78

Cation Exchange Chromatography

Cation exchange chromatography is carried out on an SP Sepharose high performance column (Pharmacia XK 26/20, 70 ml bed volume) equilibrated in 10 mM sodium acetate pH 4.0 (Buffer B). The reaction mixture is diluted 10× with buffer B and loaded onto the column at a flow rate of 5 mL/min. Next the column is washed with 5 column volumes of buffer B, followed by 5 column volumes of 12% buffer C (10 mM acetate pH 4.5, 1 M NaCl). Subsequently, the PEG-hGH species are eluted from the column with a linear gradient of 12 to 27% buffer C in 20 column volumes. The eluant is monitored at 280 nm and 10 mL fractions were collected. Fractions are pooled according to extent of pegylation (mono, di, tri etc.), exchanged into 10 mM acetate pH 4.5 buffer and concentrated to 1-5 mg/mL in a stirred cell fitted with an Amicon YM10 membrane. Protein concentration of pool is determined by A280 nm using an extinction coefficient of 0.78.

### Example 5

Biochemical Characterization

The purified pegylated hGH pools were characterized by non-reducing SDS-PAGE, non-denaturing Size Exclusion Chromatography, and peptide mapping.

Size Exclusion High Performance Liquid Chromatography (SEC-HPLC)

Non-Denaturing SEC-HPLC

The reaction of Methoxy-PEG of various attachment chemistries, sizes, linkers, and geometries with hGH, anion exchange purification pools and final purified products were assessed using non-denaturing SEC-HPLC. Analytical non-denaturing SEC-HPLC was carried out using a column, Superdex 200 7.8 mm×30 cm, (Amersham Bioscience, Piscataway, N.J.) in 20 mM Phosphate pH 7.2, 150 mM NaCl at a flow rate of 0.5 mL/minute (optionally Tosohaas G4000PWXL Amersham Bioscience, Piscataway, N.J.). PEGylation greatly increases the hydrodynamic volume of the protein resulting in a shift to an earlier retention time. New species were observed in the PEG aldehyde hGH reaction mixtures along with unmodified hGH. These PEGylated and non-PEGylated species were separated on Q-Sepharose chromatography, and the resultant purified mono PEG-Aldehyde hGH species were subsequently shown to elute as a single peak on non-denaturing SEC (>95% purity). The Q-Sepharose chromatography step effectively removed free PEG, hGH, and multi PEGylated hGH species from the mono-Pegylated hGH

Denaturing SEC-HPLC

The reaction of the butyrylaldehyde polyethylene glycols with hGH, anion exchange purification, and final purified products are assessed using denaturing SEC-HPLC. Analytical denaturing SEC-HPLC is carried out using a Tosohaas 3000SWXL column 7.8 mm×30 cm (Tosohaas Pharmacia Biotech, Piscataway, N.J.) in 100 mM Phosphate pH 6.8, 0.1% SDS at a flow rate of 0.8 mL/minute. PEGylation greatly increases the hydrodynamic volume of the protein resulting in a shift to an earlier retention time. PEGylated and non-PEGylated species are separated on Q-Sepharose chromatography

SDS PAGE/PVDF Transfer

SDS-PAGE was used to assess the reaction PEG butyrylaldehyde with hGH and the purified final products. SDS-PAGE was carried out on 1 mm thick 10-20% Tris tricine gels (Invitrogen, Carlsbad, Calif.) under reducing and non-reducing conditions and stained using a Novex Colloidal Coomassie™ G-250 staining kit (Invitrogen, Carlsbad, Calif.). Bands are blotted onto PVDF membrane for subsequent N-terminal sequence identification.

Analytical Anion Exchange HPLC

The PEG butyrylaldehyde/hGH reaction mixture, anion exchange purification fractions, and final purified products were assessed using analytical anion exchange HPLC. Analytical anion exchange HPLC was carried out using a Tosohaas Q5PW or DEAE-PW anion exchange column, 7.5 mm×75 mm (Tosohaas Pharmacia Biotech, Piscataway, N.J.) in 50 mM Tris ph 8.6 at a flow rate of 1 mL/min. Samples were eluted with a linear gradient of 5-200 mM NaCl.

N-terminal Sequence and Peptide Mapping

Automated Edman degradation chemistry was used to determine the NH₂-terminal protein sequence. An Applied Biosystems Model 494 Procise sequencer (Perkin Elmer, Wellesley, Mass.) was employed for the degradation. The respective PTH-AA derivatives were identified by RP-HPLC analysis in an on-line fashion employing an Applied Biosystems Model 140C PTH analyzer fitted with a Perkin Elmer/Brownlee 2.1 mm i.d. PTH-C18 column.

Tryptic digest were performed at a concentration of 1 mg/mL and typically 50 ug of material is used per digest. Trypsin was added such that the trypsin to PEG-hGH ratio was 1:30 (w/w). Tris buffer was present at 30 mM, pH 7.5. Samples were incubated at room temperature for 16±0.5 hours. Reactions were quenched by the addition of 50 μL of 1N HCl per mL of digestion solution. Samples were diluted, prior to placing the samples in the autosampler, to a final concentration of 0.25 mg/ml in 6.25% acetonitrile. Acetonitrile was added first (to 19.8% acetonitrile), mixed gently, and then water is added to final volume (four times the starting volume). Extra digestion solution may be removed and stored for up to 1 week at −20° C.

A Waters Alliance 2695 HPLC system was used for analysis, but other systems should produce similar results. The column used was an Astec C-4 polymeric 25 cm×4.6 mm column with 5 μm particles. Experiments were conducted at ambient temperature on a typical load of 50 μg of protein per sample. Buffer A is 0.1% trifluoroacetic acid in water; buffer B is 0.085% trifluoroacetic acid in acetonitrile. The gradient was as follows:

| Time | A % | B % | C % | D % | Flow | Curve |
|---|---|---|---|---|---|---|
| 0.00 | 0.0 | 0.0 | 100.0 | 0.0 | 1.000 | 1 |
| 90.00 | 0.0 | 0.0 | 55.0 | 45.0 | 1.000 | 6 |
| 90.10 | 0.0 | 0.0 | 0.0 | 100.0 | 1.000 | 6 |
| 91.00 | 0.0 | 0.0 | 0.0 | 100.0 | 1.000 | 6 |
| 91.10 | 0.0 | 0.0 | 100.0 | 0.0 | 1.000 | 6 |
| 95.00 | 0.0 | 0.0 | 100.0 | 0.0 | 1.000 | 6 |

The column is heated to 40° C. using a heat jacket. Peaks were detected using a Waters 996 PDA detector collecting data between 210 and 300 nm. The extracted chromatogram at 214 nm was used for sample analysis.

Tryptic maps were performed for hGH, 40K branched PEG-aldehyde, and 40K branched butyrylaldehyde (FIG. 1). The N-terminal tryptic fragment was referred to as T-1. The percent T-1 present compared to unPEGylated hGH is shown in Table 2. This data suggest that 90% of the PEG modification is at the N-terminus with remainder apparently linked to one of several possible lysine residues using PEG-aldehyde compared to greater than 98% at the N-terminus using PEG-butyrylaldehyde.

TABLE 2

| | | % T-1 present |
|---|---|---|
| | % T-1 | compared to |
| | present | unPEGylated hGH |
| hGH | 28.0% | |
| PEG-aldehyde/hGH | 2.6% | 9.2% |
| PEG- | | |
| butyrylaldehyde/hGH | 0.3% | 1.2% |

### Example 6

Pharmacodynamic Studies

Rat weight Gain

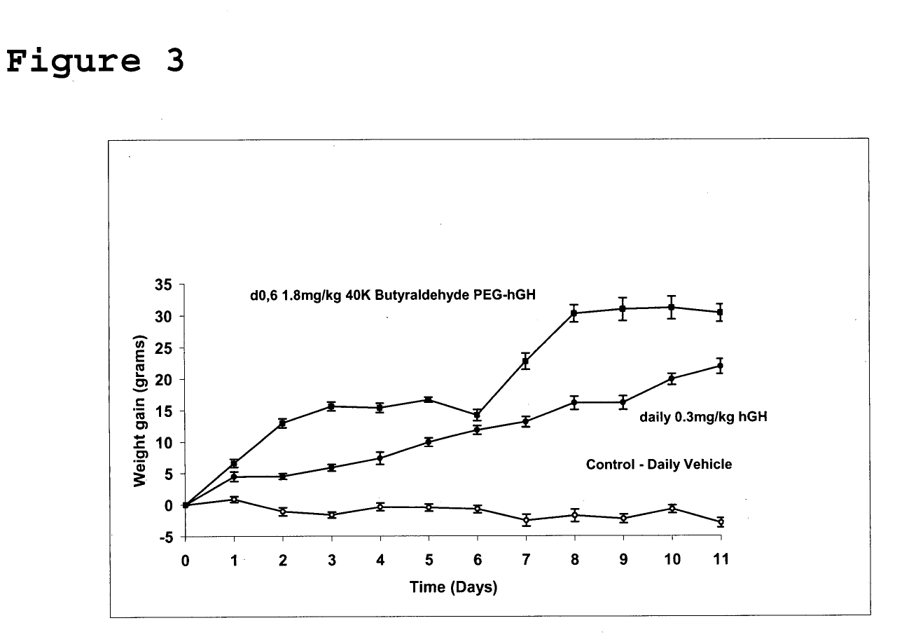

Female Sprague Dawley rats, hypophysectomized at Taconic Labs, were prescreened for growth rate for a period of 7 to 11 days. Rats were divided into groups of eight. Group 1 consisting of rats given either daily or day 0 and day 6 subcutaneous dose of vehicle. Group 2 were given daily subcutaneous dose of GH (30 μg/rat/dose). Group 3 were given subcutaneous doses of GH on day 0 and day 6(180 μg/rat/dose). Group 4 were given subcutaneous doses of PEG-GHs on day 0,6 (180 μg/rat/dose). Hypophysectomized rats were monitored for weight gain by weighing at least every other day during the study. FIGS. 3 & 4.

Rat Tibia Length

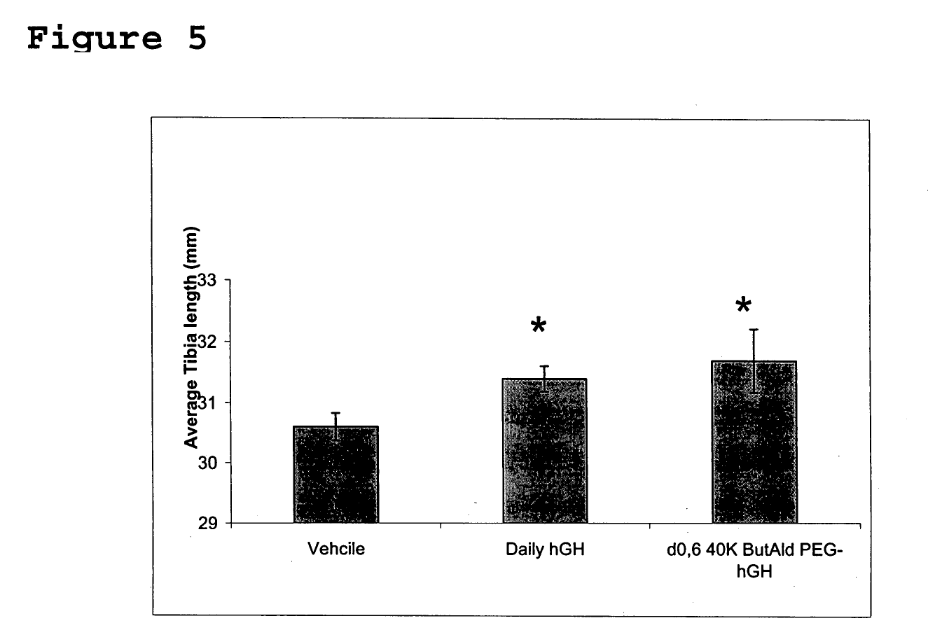

Animals in 11 Day weight gain studies at day 11 were sacrificed, left tibias were removed and X-rayed and bone lengths were measured using a caliper. FIG. 5

IGF-1 Studies

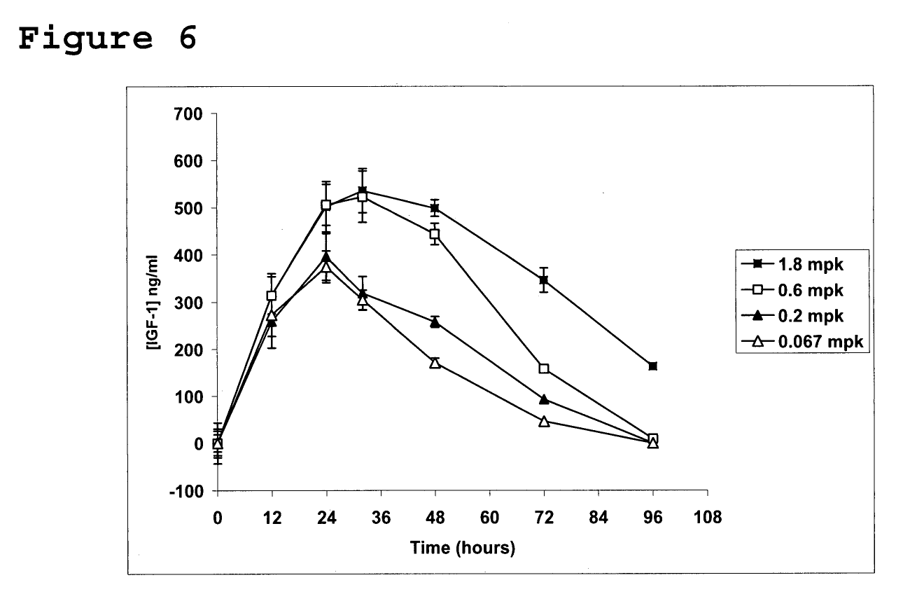

Animals from six day weight gain studies were used. Blood samples were taken at the various times during the study and the serum IGF-1 levels determined by ELISA. FIG. 6

Pharmacokinetic Studies

Pharmacokinetic studies were conducted in normal, cannulated Sprague-Dawley male rats. Injections were made as a single subcutaneous bolus of 100 μg/kg/rat GH or PEG-GH using six rats per group. Blood samples were taken over one to five days as appropriate for assessment of relevant PK parameters. GH and PEG-GH blood levels were monitored at each sampling using immuno-assay.

hGH Immunoassay

hGH and pegylated hGH protein concentration levels in mouse and cynomolgus monkey plasma were determined using the hGH AutoDELFIA kit fluorescence immunoassay (PerkinElmer). Rat and human IGF-1 levels were monitored by immunoassay kit (Diagnostic System Laboratories)

Non-Compartmental Pharmacokinetic Properties for hGH-PEG Conjugate of Example 1 in Non-Human Primates.

The hGH-PEG conjugate of Example 1 was administered to cynomolgus monkeys as 0.18 mg/kg intravenous (iv) or subcutaneous (sc) bolus injections (Table 3). PK parameters were determined using mean data for n=3 animals. Plasma concentrations were measured using the AutoDELFIA kit fluorescence immunoassay (PerkinElmer) and a standard curve pre-determined for the PEG-GH conjugate.

TABLE 3

| | iv 0.18/sc |
|---|---|
| Dose (mg/kg) | 0.18 |
| CL, iv (ml/hr/kg) | 0.8 |
| Vss (ml/kg) | 28.0 |
| T1/2, iv (hr) | 25.0 |
| T1/2, sc (hr) | 61.2 |
| SC AUC (μg/ml * hr) | 195 |
| SC Bioavailability (%) | 84 |
| Tmax, sc (hr) | 32 |

What is claimed is:

1. A PEG conjugate having the structure of the Formula 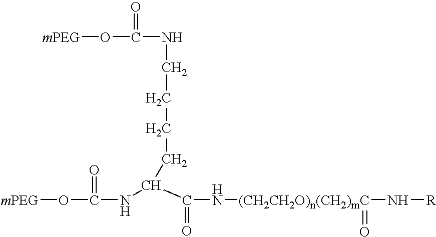
wherein
n is an integer between 1 and 10;
m is an integer between 1 and 10;
R is human growth hormone or methionyl human growth hormone.

2. The PEG conjugate of claim 1 having the structure 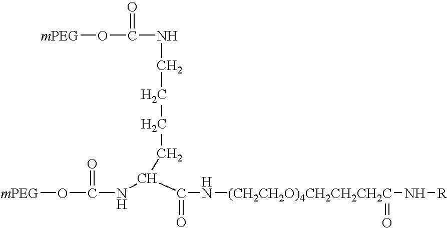
wherein R is human growth hormone or methionyl human growth hormone.

3. The conjugate of claim 2 wherein said human growth hormone comprises an amino acid sequence of SEQ ID NO:1.

4. The conjugate of claim 2 wherein said human growth hormone consists of an amino acid sequence of SEQ ID NO:1.

5. The human growth hormone-PEG conjugate of claim 3 or 4 wherein greater than 90% of said polyethylene glycol is conjugated to an amino-terminal phenylalanine of the amino acid sequence of SEQ ID NO:1.

6. The human growth hormone-PEG conjugate of claim 3 or 4 wherein greater than 95% of said polyethylene glycol is conjugated to an amino-terminal phenylalanine of the amino acid sequence of SEQ ID NO:1.

7. The human growth hormone-PEG conjugate of claim 5 wherein each mPEG has a molecular weight of about 20 kDa.

8. The human growth hormone-PEG conjugate of claim 6 wherein each mPEG has a molecular weight of about 20 kDa.

9. A composition comprising the human growth hormone-PEG conjugate of claim 7 and at least one pharmaceutically acceptable carrier.

10. A method of treating a patient having a growth or development disorder comprising administering to said patient a therapeutically effective amount of the human growth hormone-PEG conjugate of claim 7.

11. The method of claim 10 wherein said growth or development disorder is Growth Hormone Deficiency (GHD).

12. The method of claim 10 wherein said growth or development disorder is Turner's syndrome.

13. The method of claim 10 wherein said growth or development disorder is Chronic Renal Insufficiency.

14. The method of claim 10 wherein said growth or development disorder is small for gestational age (SGA).

15. A composition comprising the human growth hormone-PEG conjugate of claim 8 and at least one pharmaceutically acceptable carrier.

16. A method of treating a patient having a growth or development disorder comprising administering to said patient a therapeutically effective amount of the human growth hormone-PEG conjugate of claim 8.

17. The method of claim 16 wherein said growth or development disorder is Growth Hormone Deficiency (GHD).

18. The method of claim 16 wherein said growth or development disorder is Turner's syndrome.

19. The method of claim 16 wherein said growth or development disorder is Chronic Renal Insufficiency.

20. The method of claim 16 wherein said growth or development disorder is small for gestational age (SGA).


SEQUENCE LISTING (1 sequence)

SEQ ID NO: 1 - protein, 191 aa, Homo sapiens
FPTIPLSRLFDNAMLRAHRLHQLAFDTYQEFEEAYIPKEQKYSFLQNPQTSLCFSESIPTPSNREETQQKSNLELLRISLLLIQSWLEPVQSLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF